(19) United States
(12) Patent Application Publication
(10) Pub. No.: US 2023/0131055 A1
(43) Pub. Date: Apr. 27, 2023

(54) Personalized Heartbeat Simulation Apparatus for Soothing Infants

(71) Applicant: Neal Arjun Prakash, San Jose, CA (US)

(72) Inventor: Neal Arjun Prakash, San Jose, CA (US)

(21) Appl. No.: 17/512,667

(22) Filed: Oct. 27, 2021

(51) Int. Cl. A61M 21/02

(52) U.S. Cl. CPC A61M 21/02; A61M 2021/0027

(57) ABSTRACT

Existing infant warming solutions comprise a bedding element, a thermoregulation element and an element to produce sounds beneficial for the infant. The most beneficial among these sounds is the heartbeat of the infant's own mother. Capturing the heartbeat well enough to enable recording and accurate, realistic replay usually requires the use of a stethoscope-like instrument by a trained person and is an error-prone process for an untrained one, making it impractical for mass use. This invention improves the state-of-the-art by using an easy-to-capture but imperfect signal, for example, from a photoelectric sensor placed on the mother's finger, to still produce a realistic simulated heartbeat sound with the mother's heartrate. This makes the invention a suitable choice for infant warming solutions intended for mass use. The invention comprises an analog signal sampling module, a logic and memory module, and a sound replay module for the audible replay of electronically stored sounds.

### CROSS REFERENCE TO RELATED APPLICATIONS

Not Applicable

### STATEMENT REGARDING FEDERALLY SPONSORED RESEARCH

Not Applicable

### FIELD OF THE INVENTION

The present invention relates generally to heartbeat simulation devices for comforting infants during periods of separation from the mother.

### BACKGROUND OF THE INVENTION

Prior related art has several instances of devices designed to provide part or all of what is now referred to as “Kangaroo Care” to infants during the periodic absence of the parent. “Kangaroo Care” is the holding of an infant on the chest of a parent for extended periods of time as part of a daily caregiving routine, and is a method proven to reduce the risk of neonatal mortality and to improve sleep and neurodevelopment in infants. See, for example, U.S. Pat. No. 6,918,770, “Infant Nurturing Medical Device”, U.S. Pat. No. 7,475,441, “Baby Comforting Apparatus”, and U.S. Pat. No. 10,201,236, “Infant Soothing System”. Most of these devices allow for the provision of, or in some cases provide, a pre-recorded or simulated heartbeat, since the mother's heartbeat is known to be a key component of the “Kangaroo Care” experience.

Prior art also has at least one device for artificial heart-beat generation—U.S. Pat. No. 3,994,282, “Heart-beat Electronic Simulator”, but such heartbeats lack the audibly detectable individual variations of the infant's own mother's heartbeat. U.S. Pat. No. 6,004,259, “Baby Calmer Kit Using Mother's Heartbeat Sound”, describes a device that records the mother's own heartbeat using a stethoscope-like system and then replays it when needed, but stethoscope usage is error-prone for untrained people, and is an unacceptable limitation that must be addressed for our need.

Separately, prior art also includes devices to sense heartbeat using photoelectric devices placed in contact with the skin. See, for example, U.S. Pat. No. 4,353,152, “Pulse Rate Monitor” and U.S. Pat. No. 9,237,855, “Wearable Heart Rate Monitor”. At least one device also exists to extract the interval between heartbeats in an analog heartbeat signal, U.S. Pat. No. 4,364,397, “Apparatus for Monitoring the Rhythm of a Patient's Heartbeat”. However, they are used for purposes different from ours, and do not solve the problem of producing a realistic audible heartbeat from the sensed heartbeat signal.

### BRIEF SUMMARY OF THE INVENTION

The product of which this invention is a part is one that consists of a thermoregulating swaddling blanket and heartbeat module to provide a “Kangaroo Care” experience to infants during the periodic absence of the mother. The product is targeted at people in resource limited settings, creating a need that the heartbeat module be an easy-to-use device that simulates or replays the mother's heartbeat.

Existing solutions for the heartbeat module either use a stethoscope-like device to capture an accurate heartbeat signal that can then be stored and replayed, or expect that the mother provide a pre-recorded heartbeat, or artificially create heartbeat-like vibrations. Stethoscope-like devices are error-prone when used by untrained people like our target audience, who also do not have the resources to provide a pre-recorded heartbeat. The simulated heartbeats of artificial heart-beat generators do not have the detectably individual variations of the infant's own mother, especially variations in the heartrate.

To overcome these problems, the chosen design for the heartbeat module uses an easy-to-use photoelectric pulse sensor, that can be wrapped around the finger of the mother, to capture a heartbeat signal. This signal is delivered to our invention, a personalized heartbeat simulation apparatus within the heartbeat module. The invention comprises an analog signal sampling module that receives the electronic heartbeat signal from the pulse sensor, a logic and memory module, and a sound replay module for the audible replay of electronically stored sounds.

The logic and memory module has two modes. In “record” mode, it uses the sampling module to detect and store in its memory the duration of each interval between successive heartbeats. The number of intervals recorded span a few minutes. In “replay” mode, it sequentially “replays” the recorded intervals by simply waiting for the duration of each interval, and at the end of each interval, it triggers the sound replay module to play one of several heartbeat sounds pre-loaded in the sound replay module. The net effect is that the listener hears a heartbeat at the rate implied by the stored intervals. The sound replay module also has a selector switch to select which heartbeat sound(s) get played. When the logic module has exhausted all recorded intervals, it simply starts over from the beginning, allowing the simulated heartbeat to continuously play for hours.

### BRIEF DESCRIPTION OF THE SEVERAL VIEWS OF THE DRAWING

FIG. 1 shows a heartbeat module, with the preferred embodiment of the personalized heartbeat simulation apparatus in “replay” mode, being used with an infant.

FIG. 2 shows a heartbeat module, with the preferred embodiment of the personalized heartbeat simulation apparatus in “record” mode, being used to record the heartbeat of the mother using a pulse sensor.

FIG. 3 shows a functional diagram of the personalized heartbeat simulation apparatus.

FIG. 4 shows a circuit diagram of the preferred embodiment of the personalized heartbeat simulation apparatus

FIG. 5 shows the electric signal delivered by the pulse sensor to the heartbeat module with the personalized heartbeat simulation apparatus

FIG. 6 shows a flow chart for the operations performed by the logic and memory module to realize the “record” mode of the personalized heartbeat simulation apparatus

FIG. 7 shows a flow chart for the operations performed by the logic and memory module to realize the “replay” mode of the personalized heartbeat simulation apparatus

### DETAILED DESCRIPTION OF THE INVENTION

Our invention is used as part of a product to provide a “Kangaroo Care” experience to infants during the periodic absence of the mother. Such products typically comprise a thermoregulating swaddling blanket and a heartbeat module to simulate or replay the heartbeat of the mother. Existing solutions for the heartbeat module either use a stethoscope-like device to capture an accurate heartbeat signal that can then be stored and replayed, or expect that the mother provide a pre-recorded heartbeat, or artificially create heartbeat-like vibrations. Stethoscope-like devices are error-prone when used by untrained people like our target audience, who live in resource limited settings around the world. These people also do not have the resources to provide a pre-recorded heartbeat. The simulated heartbeats of artificial heart-beat generators do not have the detectably individual variations of the infant's own mother, especially variations in the heartrate.

Our invention is a personalized heartbeat simulation apparatus within the heartbeat module. It records an imperfect heartbeat signal from any convenient heartbeat sensing device for a few minutes, for example an easy-to-use pulse sensor wrapped around the mother's finger and transforms that into a realistic heartbeat that can be played for hours.

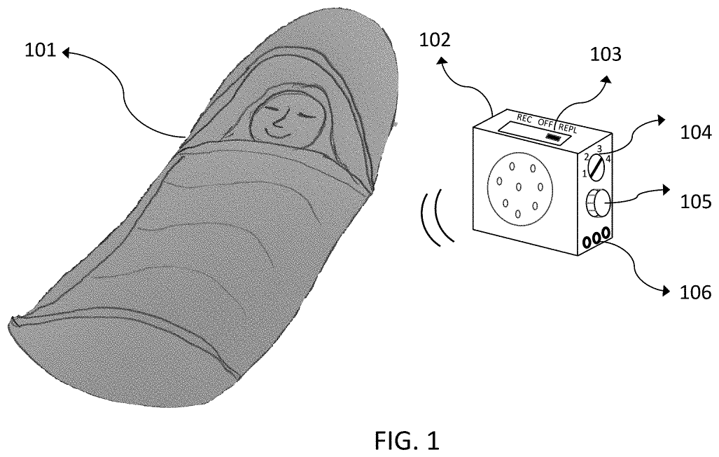

The invention has two modes of use, a “record” mode and a “replay” mode. FIG. 1 shows the external view of the preferred embodiment of our invention 102 placed in “replay” mode next to a swaddled infant 101. The preferred embodiment of our invention has three external controls and one input socket. The first is a mode control switch 103 with two settings “record” and “replay” with an “off” position in between. The second is a heartbeat sound selecting rotary switch 104 that is used to select one of several pre-recorded single heartbeat sounds that is played in “replay” mode. The third is a volume control knob 105 that controls the loudness of the heartbeat sound that is played in “replay” mode. The input socket 106 is where the leads of a pulse sensor are plugged in “record” mode.

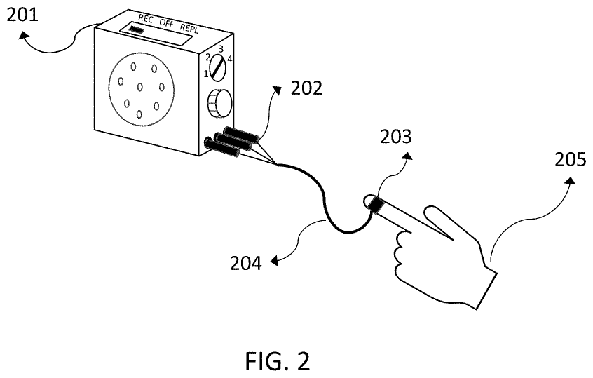

FIG. 2 shows how the preferred embodiment of our invention 201 is used in “record” mode. The infant's mother wraps the sensing part 203 of the pulse sensor 204 around the index finger of her hand 205. The leads of the pulse sensor 202 are plugged into the input socket of the preferred embodiment of our invention. The infant's mother switches the mode control switch to the “record” position from the “off” position to begin recording the duration of the intervals between her heartbeats. To complete the recording, she moves the switch back to the “off” position. Subsequently, when the pulse sensor is removed, and the mode control switch is moved to the “replay” position, the heartbeat sound indicated by the heartbeat sound selecting rotary switch is played at exactly the recorded intervals between the mother's recorded heartbeats. This simulates the mother's heartbeat for the duration of the recording, and then automatically restarts from the beginning, thereby continuously simulating the mother's heartbeat for any desired duration.

This invention represents an improvement over existing heartbeat modules as it can play a realistic personalized heartbeat by recording an error-free and easy-to-use but imperfect heartbeat signal like that obtained from a pulse sensor, as is used in the preferred embodiment of our invention. In comparison, simply playing the signal recorded by a pulse sensor does not produce a realistic heartbeat sound. This is because ordinary pulse sensors are unable to capture all the component frequencies in a heartbeat and are consequently only approximate, imperfect heartbeat sensors.

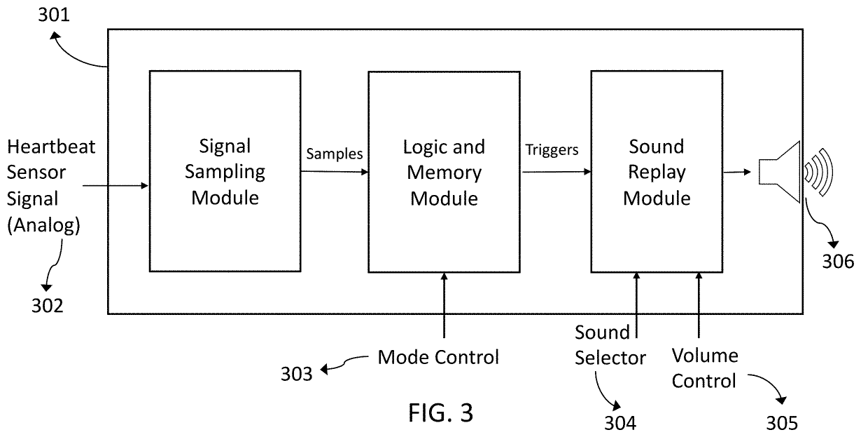

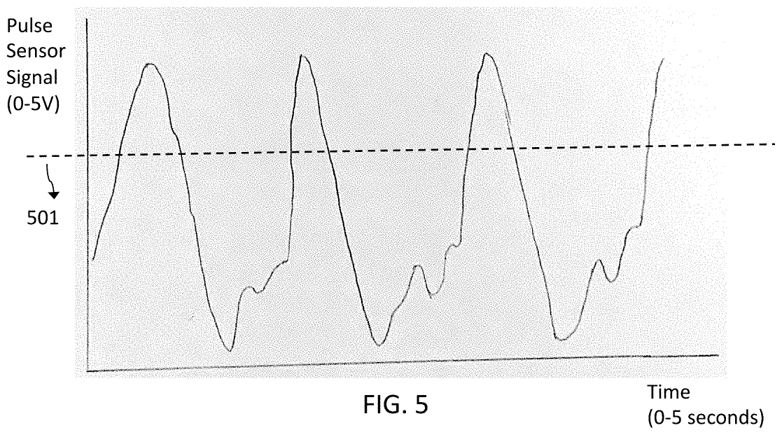

FIG. 3 shows a functional diagram of the internal implementation of the personalized heartbeat simulation apparatus 301. The signal from the pulse sensor is shown as an input to it 302. The signal sampling module samples the incoming signal, an example of which is shown in FIG. 5, at a rate several times higher than the fastest heartrate possible and delivers these samples to the logic and memory module. The mode control switch 303 connects to the logic and memory module and sets it in “record” or “replay” mode.

In “record” mode, the logic and memory module detects the first rising edge of each heartbeat using the samples continuously being taken by the signal sampling module and calculates and stores the duration of the intervals between successive rising edges in its memory. In “replay” mode, the logic and memory module triggers the sound replay module at the previously recorded intervals. The sound replay module is pre-loaded with one or more heartbeat sounds, and both the heartbeat sound selecting rotary switch 304 and the volume control knob 305 connect to it. When triggered by the logic and memory module it plays the selected sound on the speaker 306 at the set volume.

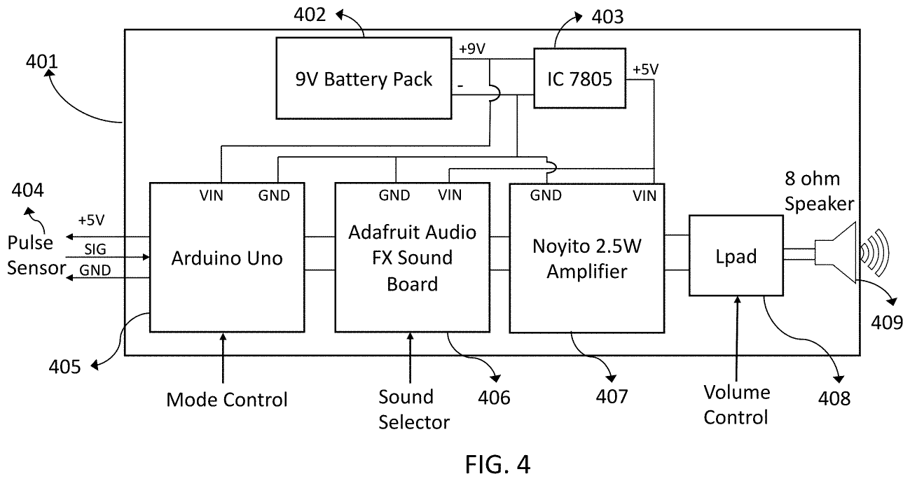

FIG. 4 shows the actual internal implementation of the preferred embodiment of the personalized heartbeat simulation apparatus 401. An Arduino Uno microcontroller 405 is used to perform the functions of the signal sampling module and the logic and memory module. The function of the sound replay module is performed by an Adafruit Audio FX Sound Board 406, a Noyito 2.5-watt Amplifier 407 and an Lpad 408. The heartbeat sound selecting rotary switch connects to the sound board, and the volume control to the Lpad. The speaker used is an 8-ohm speaker 409. On being triggered by the Arduino Uno, the sound board plays the selected sound, which is amplified by the amplifier and sent to the speaker through the Lpad for volume control. The entire circuit is powered by a battery pack 402 which has two rechargeable 9V batteries in parallel. Its 9V output is used to power the Arduino Uno and is regulated to 5V through the IC 7805 regulator chip 403. The output of the chip supplies 5V power to the sound board and the amplifier. The pulse sensor connects to the Arduino Uno using three leads 404, where two of them are for supplying 5V power to the pulse sensor, and one of them is to receive the signal from the sensor.

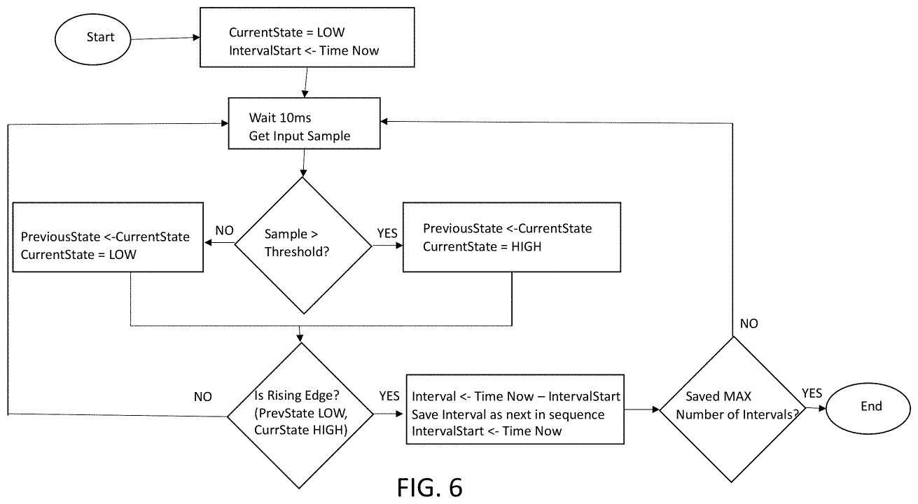

FIG. 6 shows a flowchart of the operations performed by the logic and memory module to realize the “record” mode. The samples taken of the input signal, of which an example is shown in FIG. 5, are judged as “high” or “low” using a suitable threshold 501 in the same figure. As shown in the flowchart, samples are continuously taken and judged as “high” or “low” every 10 millseconds. The variables PreviousState and CurrentState are used to judge whether a rising edge has just been seen, and if so, the interval between previous rising edge and the current one is calculated using the current time and the variable IntervalStart. Intervals are stored in sequence until a predefined maximum number of intervals have been stored.

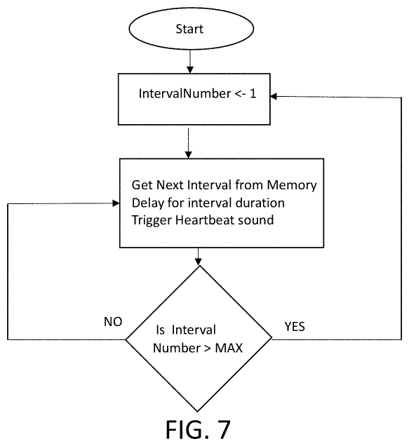

FIG. 7 shows a flowchart of the operations performed by the logic and memory module to realize the “replay” mode. The sequence of intervals stored in memory during the record mode are retrieved one-by-one and for each one, after waiting for the duration of the interval, a heartbeat sound is triggered. When the entire sequence is exhausted, it automatically restarts from the beginning allowing play for an unlimited amount of time.

With respect to the above description then, it is to be realized that the optimum dimensional relationship for the various components of the invention described above and in FIGS. 1 through 6, include variations in size, materials, shape, form, function, and manner of operation, assembly and use, and are deemed readily apparent and obvious to one skilled in the art, and all equivalent relationships to those illustrated in the drawings and described in the specification are intended to be encompassed by the invention.

It shall be noted that those skilled in the art will readily recognize numerous adaptations and modifications which can be made to the various embodiments of the present invention which will result in an improved invention, yet all of which fall within the spirit and scope of the present invention as defined in the following claims. Accordingly, the invention is to be limited by the scope of the following claims and their equivalents.

The inventor claims:

1. A personalized heartbeat simulation apparatus comprising:
A signal sampling module, a logic and memory module, a sound replay module, and a control system;
Wherein the signal sampling module continuously samples an electrical signal from a signal source and delivers the samples to the logic and memory module;
Wherein the logic and memory module can detect when samples delivered to it cross a defined threshold, and store the duration of the interval between successive crossings;
Wherein the logic and memory module has a plurality of modes of operation including a recording mode and a replay mode;
Wherein the logic and memory module can trigger the sound replay module to play one or more sounds after waiting for the duration of each previously stored interval;
Wherein the sound replay module has a storage device with a plurality of stored digitally represented sound files;
Wherein the sound replay module plays audible sounds from its store of sound files on being triggered;
Wherein the sound replay module has a plurality of controls including one to select which sound file is to be replayed and one to control the volume of the sounds;
Wherein the control system provides a plurality of external controls including all the controls of the logic and memory module and the sound replay module.

2. The personalized heartbeat simulation apparatus according to claim 1:
Wherein the signal source of the signal sampling module is a heartbeat sensing device.

3. The personalized heartbeat simulation apparatus according to claim 1:
Wherein each digitally represented sound file stored in the sound replay module is the sound of a single heartbeat.
